(19) United States
(12) Patent Application Publication
(10) Pub. No.: US 2024/0093150 A1
(43) Pub. Date: Mar. 21, 2024

(54) IMPROVED T-CELLS FOR CANCER THERAPY USING AMINO ACID STARVATION PATHWAYS

(71) Applicant: UNIVERSITY HEALTH NETWORK, Toronto (CA)

(72) Inventors: Pamela OHASHI, Toronto (CA); Sam SAIBIL, Toronto (CA); Michael ST. PAUL, Richmond Hill (CA)

(21) Appl. No.: 18/263,658

(22) PCT Filed: Jan. 18, 2022
(86) PCT No.: PCT/CA2022/050067

§ 371 Date: Jul. 31, 2023

Related U.S. Application Data
(60) Provisional application No. 63/144,267, filed on Feb. 1, 2021.

(51) Int. Cl. C12N 5/0783; A61K 35/17; A61P 35/00

(52) U.S. Cl. CPC C12N 5/0636; A61K 35/17; A61P 35/00; C12N 2501/727

(57) ABSTRACT

There is described herein a method for improving the anti-cancer properties of T-cells, the method comprising: providing a population of T-cells; and culturing the T-cells in an environment that activates the GCN2 pathway.

### CROSS REFERENCE TO RELATED APPLICATIONS

This application claims priority to U.S. Provisional Application No. 63/144,267 filed on Feb. 1, 2021, and which is herein incorporated by reference in its entirety.

### FIELD OF THE INVENTION

The invention relates to T-Cells for cancer therapy, including methods for improving the same.

### BACKGROUND OF THE INVENTION

Immune therapies, ranging from checkpoint blockade antibodies to cell therapy with T cells expressing chimeric antigen receptors (CARs), have revolutionized the treatment of cancer. Despite this success, many patients still do not respond to immunotherapy. A major barrier to the success of immune-based treatments are the numerous immunosuppressive factors within the tumor-microenvironment (TME) (Frey, 2015). In addition to cytokines and regulatory cell populations, multiple metabolic factors have been identified that potentially inhibit T cell function in the TME (Ngwa et al., 2019). These factors include hypoxia (Petrova et al., 2018), increased concentrations of lactic acid (Brand et al., 2016) as well as increased concentrations of potassium released from necrotic cells (Eil et al., 2016). Additionally, consumption of key metabolic substrates such as glucose (Chang et al., 2015) and oxygen (Najjar et al., 2019) by metabolically active tumor cells can limit the availability of these nutrients to T cells and constrain T cell activity. Similarly, depletion of the important amino acids tryptophan and arginine via the expression of the enzymes indole 2,3,-dioxygenase (IDO) and arginase in the TME have been proposed to impair T cell function (Lemos et al., 2019). It has also been suggested that low intratumoral levels of other amino acids, such as glutamine, results in impaired T cell function (Renner et al., 2017). Conversely, it has recently been reported that pharmacological inhibition of glutamine metabolism limits tumor cell proliferation whilst paradoxically enhancing the function of T cells in the TME (Leone et al., 2019). Moreover, preconditioning with transient glucose deprivation has been found to enhance the efficacy of T cells used for cell therapy in murine models (Klein Geltink et al., 2020). Thus, the effects of nutrient deprivation on T cells, and particularly on previously activated T cells, remains to be fully elucidated.

### SUMMARY OF THE INVENTION

In an aspect, there is provided a method for improving the anti-cancer properties of T-cells, the method comprising: providing a population of T-cells; and culturing the T-cells in an environment that activates the GCN2 pathway.

In an aspect, there is provided a population of anti-cancer T-cells produced by the methods described herein.

In an aspect, there is provided a use of the population of anti-cancer T-cells described herein, in the preparation of a medicament for the treatment of cancer.

In an aspect, there is provided a method of treating a patient with cancer, the method comprising administering to the patient the population of anti-cancer T-cells described herein.

In an aspect, there is provided a method of treating a patient with cancer, the method comprising administering to the patient a GCN2 pathway agonist.

### BRIEF DESCRIPTION OF FIGURES

These and other features of the preferred embodiments of the invention will become more apparent in the following detailed description in which reference is made to the appended drawings wherein:

FIG. 1. Amino acid starvation response enhances T cell effector function and oxidative metabolism. (a) Diagram illustrating experimental protocol. P14 CD8+ T cells were activated with peptide pulsed bone marrow dendritic cells in complete media. After three days of activation, cells were expanded with IL-2 for another 4 days in arginine replete or deficient media. (b) Cytokine production in CD8+ T cells expanded in the presence or absence of arginine. (c) IFN-γ mean fluorescence intensity (MFI) and percent cytokine positive (n=4) CD8+ T cells cultured as indicated. (d) Seahorse analysis quantifying the oxygen consumption rate (OCR) of T cells expanded in the presence or absence of arginine (n=±SEM 3). (e) Basal OCR, extracellular acidification rate (ECAR) and OCR:ECAR ratio as determined by seahorse (n=3). (f) ATP levels in T cells (n=3). (g) Gene expression of downstream targets of GCN2 as determined by real-time PCR. Values are expressed as fold-change relative to T cells expanded in arginine (n=4). (h) CD98 expression as determined by flow cytometry as well as CD98 MFI from n=4 mice. (i) Diagram illustrating experimental protocol for halofuginone (Halo) treatment during the last 48 hours of T cell activation. w Cytokine production in Halo treated CD8+ T cells. (k) Percent cytokine positive and MFI of IFN-γ in halo-treated CD8+ T cells (n=3). (l) Granzyme B expression in halo treated cells (n=3). (m) CD98 expression in halo treated cells (n=3). (n) Seahorse analysis quantifying the OCR of Halo-treated CD8+ T cells (n=5±SEM). (o) OCR:ECAR ratio as determined by seahorse (n=5). (p) ATP levels in halo-treated T cells (n=3). Each circle represents a different mouse. Grey histograms represent fluorescent minus one (FMO) controls. Results shown are representative of at least 2-3 independent experiments or pooled from multiple independent experiments. *p<0.05, **p<0.01 as determined by two-tailed t-test. n.s.=not significant.

FIG. 2. Halofuginone promotes the transcriptional regulation of 4-1BB expression and mitochondrial metabolism. (a) Flow cytometry of halo or vehicle treated cells evaluating the expression of surface markers associated with Tcm lineage. (b-g,i,j) Total and ribosomal enriched RNA was extracted and sequenced from halo or vehicle treated CD8+ T cells from 3 mice. (b) Principal component analysis plot of Halo or Vehicle treated CD8+ T cells. (c) Volcano plot of highly up or down-regulated genes in Halo-treated cells as found in total RNA as well as those regulated translationally as determined by translational efficiency (TE). Ingenuity pathway analysis identifying the pathways most enriched in Halo-treated cells in (d) total RNA and (e) TE (all pathways were selected with p-value <0.05). Log 2 fold change levels vs vehicle of total and ribosomal RNA fractions as well as TE of genes associated with (f) the Trm cell lineage and (g) T cell co-stimulatory and inhibitory molecules. (h) Expression level of 4-1BB in halo or vehicle-treated CD8+ T cells as determined by flow cytometry (N=3±SEM). (i) FPKM levels of Bhlhe40 in halo and vehicle-treated cells as determined by RNA sequencing (N=3±SEM). a) Log 2 fold change levels vs vehicle of total and ribosomal RNA fractions as well as TE of genes associated with the electron transport chain complexes. **p<0.01 as determined by (h) two-tailed t-test or (i) ANOVA with Tukey test. n.s.=not significant.

FIG. 3. Autophagy and the CD98-mTOR axis mediate enhanced OXPHOS and IFN-γ production. (a) Amino acid levels as determined by mass spectrometry. Values represent average z-score from n=4 mice. (b) p-mTOR staining in Halo-treated CD8+ T cells. (c) p-mTOR MFI after treatment with BCH (n=3). (d) MFI of 4-1BB, Granzyme B and IFN-γ after treatment with BCH. (n=3) (e) Basal OCR after treatment with BCH (n=3). (f) Autophagy levels in Halo-treated cells (n=3). (g) Seahorse analysis quantifying OCR of T cells treated as indicated (n=3±SEM). (h) Basal OCR after treatment with 3MA (n=3). (i) IFN-γ scatter plot and a) MFI (n=3) after treatment with 3MA. (k) IFN-γ scatter plot and (l) MFI (n=3) after treatment with oligomycin. (m) Diagram illustrating the proposed mechanism. Results shown are representative of at least 2-3 independent experiments. Bars indicate mean and each circle represents an individual mouse. *p<0.05, **p<0.01 as determined by ANOVA with Tukey test or (f) two tailed t test. n.s.=not significant.

FIG. 4. Halo-treated CD8+ T cells demonstrate robust anti-tumor activity. (a) Mice bearing day 10 established EG7-OVA tumors were administered 1×10⁶ halo or vehicle treated OT-1 cells. Each line is a different mouse (n=6). (b) Survival curve from (a) representing combined survival across all experiments (n=11-12). (c) Mice bearing day 11 established B16-gp33tumors received an adoptive transfer of 0.5×10⁶ halo or vehicle treated P14 cells in conjunction with an in vivo administration of 4-1BB agonistic antibody. Each line is a different mouse (n=5). (d) Day 25 tumor size from (e) pooled from multiple independent experiments (n=10). (f) OCR seahorse curve of halo-treated human CD8+ T cells (n=4±SEM). (f-i) Human naive CD8+ T cells were activated in the presence of halo and transduced with DMF5 TCR. (f) FACs plot from a representative donor showing high TCR expression in both treatment groups. Representative histogram and pooled MFI of (g) CD98, (h) 4-1BB and (i) Granzyme B gated on TCR+CD8+ T cells as shown in (f) (n=6). Results shown are representative of at least 2 independent experiments (a,c,e,f) or pooled from multiple independent experiments (b,d,g-i). *p<0.05, **p<0.01 as determined by (b) log-rank test (d) ANOVA with Tukey test or (g-i) Mann-Whitney U test.

FIG. 5. Halo increases TNF IL-2 and IFN-γ poly-functionality. Activated CD8+ T cells were incubated with IL-2 for 4 days with either halofuginone (halo) or vehicle control added for the last 2 days of expansion. Prior to analysis, cells were re-stimulated with PMA/ionomycin in the presence of Brefeldin A for 5-6 hours. (a) Flow cytometry plot indicating gating strategy. (b) IL-2 and TNF-α expression in vehicle or halo-treated CD8+ T cells. % Cytokine positive and mean fluorescence intensity (MFI) of (c) TNF-α and (d) IL-2 in halo or vehicle-treated CD8+ T cells (n=3). (e) % of cells producing either IFN-γ, TNF-α, IL-2 or nothing. Colored bars represent the amount of cells co-producing the indicated number of cytokines. Results shown are representative of at least 3 independent experiments.

FIG. 6. Halo-treated cells are effector T cells with increased 4-BB expression. Activated CD8+ T cells were incubated with IL-2 for 4 days with either halofuginone (halo) or vehicle control added for the last 2 days of expansion and analyzed by flow cytometry to evaluate expression of (a) CD62L and CD44 (b) CD127 and CD25 or (c) multiple different surface markers associated with T cell differentiation and effector functions. Each box in (c) represents a different mouse. Results shown are representative of at least 2 independent experiments.

FIG. 7. Halo up-regulates genes involved with glycyolysis and TCA cycle but not fatty acid oxidation. Total and ribosomal RNA was extracted from halo or vehicle treated CD8+ T cells from 3 mice and sequenced. Heatmaps represent mean Log 2 fold change vs vehicle control of genes associated with (a) glycolysis (b) TCA cycle or (c) fatty acid oxidation.

FIG. 8. Halofuginone alters the T cell metabolic profile. Metabolites were extracted from vehicle or halo treated CD8+ T cells from 4 mice and analyzed with mass spectrometry. (a) Heatmap and (b) Random forest plots of top metabolites increased or decreased in halo-treated cells.

FIG. 9. mTOR, but not autophagy, regulates expression of 4-1BB and Granzyme B in halo-treated cells. Effector CD8+ T cells were incubated with Halofuginone in the presence of (a) Rapamycin or (b) 3MA for 48 hours. Cells were analyzed by flow cytometry to evaluate expression of the 4-1BB, Granzyme B and IFN-γ. Results shown are mean fluorescence intensity from 3 mice±SEM. Results shown are representative of at least 2 independent experiments. **p<0.01 as determined by two-tailed t-test, n.s. not significant.

FIG. 10. Halofuginone enhances 4-1BB and CD98 expression in human CD8+ T cells. Human CD8+ T cells were isolated from PBMC and activated in the presence of halofuginone or vehicle control. Cells were stained for (a) 4-1BB and (b) CD98 expression and analyzed with flow cytometry. Results shown are mean (a) % positive or (b) MFI from 5 healthy donors pooled from multiple independent experiments. * p<0.05, **p<0.01 as determined by two tailed t test.

### DETAILED DESCRIPTION

In the following description, numerous specific details are set forth to provide a thorough understanding of the invention. However, it is understood that the invention may be practiced without these specific details.

The manipulation of T cell metabolism to enhance anti-tumor activity is an area of active investigation. Here, we report that activating the amino acid starvation response in effector CD8+ T cells using the General Control Non-depressible 2 (GCN2) agonist halofuginone (halo) enhances oxidative metabolism and effector function in mouse and human CD8+ T cells. Further characterization revealed that halo-treated CD8+ T cells increased expression of the large neutral amino acid (LNAA) transporter CD98 as well as the co-stimulatory marker 4-1BB. Mechanistically, we identified autophagy coupled with the CD98-mTOR axis as key downstream mediators of the phenotype induced by halo treatment. The adoptive transfer of halo-treated CD8+ T cells into mice bearing well-established tumors led to robust tumor control and curative responses. The adoptive transfer of halo-treated T cells also synergized with an in vivo treatment of 4-1BB agonistic antibody to control tumor growth in a mouse model resistant to immunotherapy. These findings demonstrate that activating the amino acid starvation response with the GCN2 agonist halofuginone can enhance T cell metabolism, effector function and anti-tumor activity, thereby providing a novel strategy to enhance existing clinical immunotherapeutic approaches.

In an aspect, there is provided a method for improving the anti-cancer properties of T-cells, the method comprising: providing a population of T-cells; and culturing the T-cells in an environment that activates the GCN2 pathway.

In some embodiments, the environment includes a GCN2 pathway agonist.

In some embodiments, the GCN2 pathway agonist is a tRNA synthetase inhibitor.

In some embodiments, the GCN2 pathway agonist is selected from the GCN2 pathway agonists disclosed in [Nature Chemical Biology, Halofuginone and other febrifugine derivatives inhibit prolyl-tRNA synthetase, vol 8, March 2012, p. 311-317].

In one embodiment, the GCN2 pathway agonist is halofuginone.

In some embodiments, the GCN2 pathway agonist is added to the culture immediately following isolation of the T-cell population.

In some embodiments, the GCN2 pathway agonist is added to the culture within 2 weeks following isolation of the T-cell population.

In some embodiments, the environment is amino acid deficient or depleted.

In some embodiments, the T-cells are CD8+.

In some embodiments, the T-cells are a Tumour Infiltrating Lymphocytes.

In some embodiments, the T cells express chimeric antigen receptors (CARs).

In an aspect, there is provided a population of anti-cancer T-cells produced by the methods described herein.

In some embodiments, the population of anti-cancer T-cells is for use in the treatment of cancer.

In an aspect, there is provided a use of the population of anti-cancer T-cells described herein, in the preparation of a medicament for the treatment of cancer.

In an aspect, there is provided a method of treating a patient with cancer, the method comprising administering to the patient the population of anti-cancer T-cells described herein.

In an aspect, there is provided a method of treating a patient with cancer, the method comprising administering to the patient a GCN2 pathway agonist.

In some embodiments, the GCN2 pathway agonist is a tRNA synthetase inhibitor. Preferably, the GCN2 pathway agonist is halofuginone. In some embodiments, the GCN2 pathway agonist is selected from the GCN2 pathway agonists disclosed in [Nature Chemical Biology, Halofuginone and other febrifugine derivatives inhibit prolyl-tRNA synthetase, vol 8, March 2012, p. 311-317].

As used herein, “pharmaceutically acceptable carrier” means any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

The advantages of the present invention are further illustrated by the following examples. The examples and their particular details set forth herein are presented for illustration only and should not be construed as a limitation on the claims of the present invention.

### EXAMPLES

### Methods and Materials

Mice and Cell lines

C57BL/6 and OT-1 mice were purchased from The Jackson Laboratory and Taconic. Generation of P14 mice, which express a transgenic TCR specific for the H2-D^{b} gp33 peptide of the lymphocytic choriomeningitis virus (LCMV) was described previously (Pircher et al., 1989). All mice were maintained at the Ontario Cancer Institute animal facility according to institutional guidelines and with approval of the Ontario Cancer Institute Animal Ethics Committee. Cell lines used include the B16 melanoma expressing the LCMV Gp33 antigen (obtained from Dr. Rolf Zinkernagel) and the EG7 thymoma line expressing ovalbumin antigen (EG7-OVA—obtained from Dr. David Brooks).

### T Cell Activation

P14 or OT-1 CD8+ T cells were magnetically purified (Miltenyi Biotec) from the spleens and lymph nodes of P14 or OT-1 mice and co-cultured with LPS-matured bone marrow dendritic cells (BMDCs) pulsed with gp33 peptide from LCMV (KAVYNFA™) for P14 cells, or the ovalbumin peptide (SIINFEKL) for OT-1 cells as described in (St. Paul et al., 2020). T cells were incubated with DCs for three days in IMDM (Gibco) supplemented with 10% FCS, L-glutamine, β-mercaptoethanol, penicillin and streptomycin. After three days, cells were expanded in fresh IMDM containing IL-2 (10 ng/mL—Biolegend) for another 96 hours and subsequently used for flow cytometry or downstream assays. In experiments involving arginine depletion, the last 96 hours of cell culture was performed in arginine-deficient IMDM. In experiments involving Halofuginone (Halo), activated CD8+ T cells were IL-2 expanded in complete IMDM for 96 hours with Halo (50 ng/mL) being added for the last 48 hours of culture.

### Flow Cytometry, Antibodies and Cytokine Assays

Antibodies used for flow cytometry were purchased from eBioscience, Biolegend and BD Pharmingen. Antibody clones used were: CD8 (53-6.7), IFN-γ (XMG1.2), TNF-α (MP6-XT22), IL-2 (JES6-5H4), 4-1BB (17B5), CD98 (4F2), CD69 (H1.2F3), CD103 (2E7), CD44 (IM7), CD62L (MEL-14), pMTOR (MRRBY), Granzyme B (GB12), CD127 (A7R34), CD25 (PC61), CCR7 (4B12), KLRG1 (2F1), Sca-1 (D7), CD28 (37.51), ICOS (7E.17G9), OX40 (OX-86), SLAM (mShad150), GITR (DTA-1), Lag3 (C9B7W), Tim3 (RMT3-23), PD1 (J43) and CTLA4 (UC10-4B9). For intracellular cytokine staining, cells were re-stimulated for 5 hours with Cell-Stimulation Cocktail (eBioscience) in the presence of Brefeldin A (eBioscience), followed by staining using Cytofix/Cytoperm (BD Pharmingen). Phosphoflow was performed using BD Phospflow Perm Buffer III (BD Pharmingen) according to manufacturer's recommended protocol. Flow cytometry data was acquired on a FACSCanto II (BD) or LSR Fortessa and analyzed using FlowJo software (Tree Star).

### Metabolic Assays and Profiling

Seahorse was performed as previously described (Saibil et al., 2019). Oligomycin (1.5 μm), Etomoxir (4 μm), FCCP (1.5 μm), and Ant/Rot (0.5 μm) were injected as indicated in the figures. ATP quantification was performed using a commercial kit (Sigma) according to the recommended protocol. For metabolic profiling of Halo treated cells, mass spectrometry was performed on metabolites extracted from cell pellets. Briefly, cell pellets were washed and snap frozen in 1 ml 80% methanol. Samples were probe sonicated for 5 seconds, power level 3 (Fisher Scientific Model 100 Sonicator). 5 ul of internal standard (Isotopically labeled amino acids, 1.25 mM, PN MSK-A2-1.2, Cambridge Isotope Laboratories) was added to a 100 ul aliquot of supernatant. 10 ul of this solution was diluted in 990 ul of buffer containing 95% acetonitrile, 5% 20 mM ammonium carbonate (pH 9.8). Quality control samples (QCs) were prepared by pooling 100 μl of each sample. All samples including QCs, where then analyzed by selected reaction monitoring (SRM) using a Waters XBridge Amide 1.0×50 mm, 3.5 μm column and a 10 mM ammonium carbonate (pH 10) acetonitrile buffer system coupled with a Sciex Qtrap 5500 triple quadrupole linear ion trap tandem mass spectrometer. The data acquisition included 317 transitions. Data were captured using Analyst, version 1.6.2 software (Sciex); peak integration was performed using Skyline, version 4.1 (Pino et al., 2020). An in-house R script was used for data QC analysis and normalization (Version 3.1.2, http://www.r-project.org). Statistical analysis was performed using the MetaboAnalystR package (Chong et al., 2019).

### Pharmacologic Compounds

Halofuginone was purchased from Caymen Chemicals. Oligomycin, Etomoxir, Rapamycin and 3-MA were purchased from Sigma. Oligomycin (1 uM), Rapamycin (20 nM) and 3-MA (2.5 mM) were added to CD8+ T cells concurrent with Halofuginone.

### RNA Extraction and Real-Time PCR

RNA was extracted using an RNA extraction kit (Qiagen) according to the recommended protocol. RNA was reverse transcribed into cDNA using qScript cDNA Super Mix (Quanta) and gene expression was quantified by real-time PCR using PerfeCTa SYBR Green FastMix (Quanta) on the Applied Biosystems 7900HT using recommended parameters. Gene expression was normalized to the house keeping gene GAPDH.

### Ribosomal RNA Extraction and Sequencing

Ribosomal profiling was conducted according to the TruSeq Ribo Profile kits manual. (Note: This kit has been discontinued however the protocol and reagents used are based on a previously published protocol (Ingolia et al., 2012)). Briefly, cultured cells were incubated in 50 mg/ml cycloheximide (CHX) for 10 min and then washed in PBS containing CHX. The samples were lysed in cytoplasmic lysis buffer and clarified by centrifugation at 12,000 g for 10 min, Aliquots (100 and 200 μL) from each supernatant were generated. 100-μL aliquot of supernatant was used to extract total RNA for constructing RNA-seq libraries and 200-μL aliquot of supernatant was treated with nuclease provided by the TruSeq Ribo Profile Kit (illumina). Nuclease digestion was stopped by adding 15 μL of SUPERase-in (Thermo Fisher Scientific; AM2696). Size exclusion columns (illustra MicroSpin S-400 HR Columns) Size exclusion columns (illustra MicroSpin S-400 HR Columns; GE Healthcare; catalog no. 27-5140-01) were equilibrated with 3 mL of polysome buffer by gravity flow and spun at 600×g for 4 min. Ribosomes were isolated by applying digested lysate immediately onto the prepared size exclusion columns above (100 μL of lysate per column) and spinning them at 600×g for 2 min. Next, 10 μL 10% (wt/vol) SDS was added to the elution, and RNA with a size greater than 17 nt was isolated according to the Zymo RNA clean and concentrator kit (Zymo Research; R1017). After checking digestion quality, RNA with a size less than 200 nt was isolated according to the Zymo RNA clean and concentrator kit (Zymo Research; R1015). rRNA was depleted using the Ribo-Zero Human/Mouse/Rat kit (illumine; RS-122-2201, RS-122-2202, and RS-122-2203). After rRNA depletion, purified RNA was separated by 15% (wt/vol) TBE-urea PAGE (Thermo Fisher Scientific; EC68852BOX), and gel slices from 28 to 30 nt were excised. Ribosome footprints were recovered from the excised gel slices following the overnight elution method specified in the kit manual. After obtaining ribosome footprints above, Ribo-seq libraries were constructed according to TruSeq Ribo Profile kit manual and amplified by 13 cycles of PCR with a barcode incorporated in the primer. The PCR products were gel purified using the overnight method described by protocol.

For RNA-seq, a 100-μL aliquot of supernatant as described above was used to extract total RNA by adding 5 μL of 10% (wt/vol) SDS followed by purification using the Zymo RNA clean and concentrator kit (Zymo Research; R1017). Then, 5 μg of total RNA were subjected to rRNA depletion using Ribo-Zero Human/Mouse/Rat kit (illumine; RS-122-2201, RS-122-2202, and RS-122-2203). The rRNA-depleted RNA was used to construct sequencing libraries using the TruSeq Ribo Profile kit (illumina). The circularized cDNA was amplified by 11 cycles of PCR and gel purified using the same procedure for the Ribo-seq libraries described above. Libraries were barcoded, pooled, and sequenced in a HiSeq 2500 machine (single-end 50 bp).

### RiboSeq Analysis

For the riboseq sequencing reads, both RPF and total fractions were processed similarly. First, adapter sequences were trimmed off using cutadapt version 1.18 and removed if shorter than 15 bp ((Martin, 2011); special parameters -m 15 -q 25). Then, all remaining reads were aligned against a non-coding RNA database in order to remove any remaining reads that cannot be uniquely assigned to ribosome-translated genes. To this end, we downloaded ncRNA sequences (tRNAs, rRNAs and others) from Ensembl version 85, and aligned all reads against this ncRNA database with bowtie2 version 2.3.4.1 (parameters: -L 18; (Langmead and Salzberg, 2012)). All unaligned reads were extracted (--un parameter) and aligned against the mouse reference genome GRCm38/mm10 using STAR version 2.5.0c (parameters --outFilterMultimapNmax 1; --outFilterMismatchNoverLmax 0.05; (Dobin et al., 2013)), with the STAR-integrated read-counting method (--quantMode GeneCounts) using gene annotations downloaded from Ensembl Version 85. In order to assess the quality of the Riboseq libraries, we checked for intra-gene read distribution as well as read-length of reads uniquely aligned to the reference genome after filtering. Both metrics displayed expected distributions. Differential analysis was conducted using edgeR version 3.16.5 (Robinson et al., 2010), using glmFit and glmLRT for normalization, the exactTest function for RPF and total fractions individually and the formula (condition+protocol+condition:protocol) for translation-efficiency. Statistical results were corrected for multiple testing using the false discovery rate.

Pathway analysis was conducted using Ingenuity Pathway Analysis (IPA). To this end, all significantly differentially “expressed” genes for the RPF, total fraction or translation efficiency were used as input (thresholds of FDR <0.05; log 2FC >1.0 or <−1.0). Results were filtered for p-value <0.05, and activation z-scores are represented.

### Tumors and Immunotherapy

For EG-7 OVA experiments, 8-12 week old female C57BL/6 mice were inoculated subcutaneously with 4×10⁵ EG7-Ova cells. 10 days later, mice bearing established tumors were randomized into different groups and received 1×10⁶ Halo or Vehicle treated CD8⁺ OT-1 T cells by tail vein infusion. Tumor size was continually assessed using calipers until mice reached experimental endpoint (diameter ≥1.5 cm or severe ulceration/necrosis).

For B16 experiments, 8-12 week old female C57BL/6 mice were inoculated with 4×10⁵ B16-gp33 cells. 11 days later, mice bearing established tumors were randomized into different groups and received 0.5×10⁶ Halo or Vehicle treated CD8⁺ P14 T cells by tail vein infusion. Concurrent to T cell infusion, some mice also received 50 μg of α-4-1BB (clone 3H3 from BioXCell) by i.v. infusion. Tumor size was continually assessed using calipers until mice reached experimental endpoint (diameter ≥1.5 cm or severe ulceration/necrosis).

### Human T Cell Experiments

Peripheral blood mononuclear cells were obtained from healthy donors following institutional review board approval. Written informed consent was obtained from all donors who provided the samples. PBMCs were magnetically sorted for naïve CD8+ T cells (Miltenyi Biotec) and activated with CD3/CD28 Dynabeads (Invitrogen) at 1:1 ratio in complete IMDM for 5 days in the presence of Halo (12.5 ng/mL) or vehicle control. For DMF5 TCR transduction, purified naïve CD8+ T cells were stimulated with CD3/CD28 Dynabeads at 1:1 ratio in complete IMDM media and 100 IU/ml recombinant human IL-2. Two days after stimulation, T cells were infection with PG13-derived virus encoding DMF5 TCR and a truncated NGFR tag, separated by 2A sequences. Halofuginone (12.5 ng/mL) or vehicle control was added on days 0 and 2. Phenotype was analyzed on day 5.

### Statistical Analysis

Statistical significance was calculated using Graphpad Prism as indicated in the figure legends. p<0.05 was considered statistically significant. *p<0.05, **p<0.01, ***p<0.001.

### Results and Discussion

### Arginine Starvation Enhances CD8+ T Cell Effector Function and OXPHOS

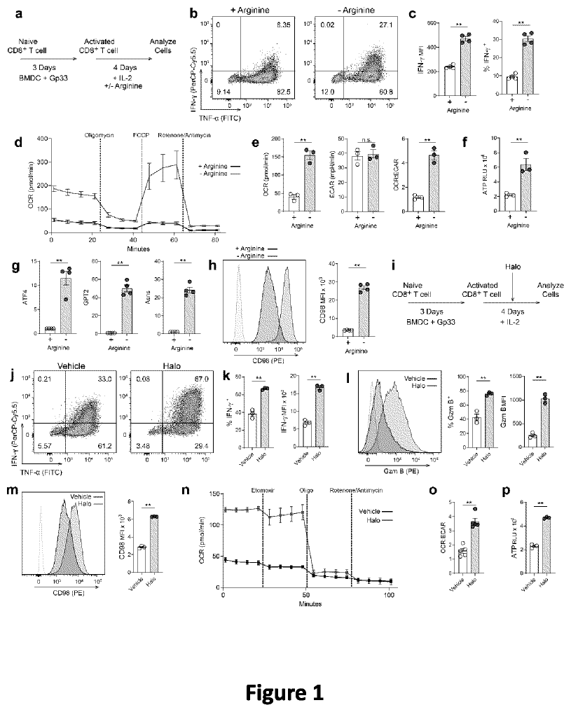

A recent report has suggested that amongst the amino acids, arginine is the most depleted within the tumoral interstitial fluid (TIF) in a murine model (Sullivan et al., 2019). Thus, to simulate the acute amino acid deprivation encountered by activated T cells upon entering the TME, we cultured activated effector CD8+ T cells in arginine free media. As described in FIG. 1A, naïve P14 CD8+ T cells were activated with peptide-pulsed mature bone marrow-derived dendritic cells (BMDC) in complete media for 3 days to generate effector CD8+ T cells. Following this, activated CD8+ T cells were expanded in IL-2 with or without arginine for an additional 4 days. Given that it has previously been reported that arginine depletion during initial T cell activation inhibits IFN-γ production (Werner et al., 2017), we reasoned a similar phenomenon would occur when previously-activated T cells were deprived of arginine. Surprisingly, we observed the opposite effects as arginine-starved effector CD8+ T cells demonstrated enhanced IFN-γ production (FIG. 1B,C) Metabolically, we found these arginine-starved cells displayed enhanced oxidative phosphorylation (OXPHOS) as indicated by an elevated basal oxygen consumption rate (OCR) in addition to an increase in spare respiratory capacity (FIG. 1D). Despite elevated OXPHOS, no change in glycolysis was observed as evident by comparable extracellular acidification rates (ECAR), thereby resulting in an increased OCR:ECAR ratio (FIG. 1E). Consistent with increased OXPHOS, we noted increased ATP levels in the arginine-starved cells (FIG. 1F). Therefore, we unexpectedly identified a metabolic stress response in which effector CD8+ T cells demonstrated enhanced effector functions and oxidative metabolism in response to starvation of a specific amino acid.

Arginine depletion during T cell activation has been demonstrated to activate the amino acid starvation response mediated by the kinase GCN2 in murine T cells (Rodriguez et al., 2007). Once activated, GCN2 phosphorylates eukaryotic Initiation Factor 2a (eIF2a) and induces reprogramming of protein translation to generally repress global protein translation whilst promoting the expression of Activating Transcription Factor 4 (ATF4) and other transcription factors involved in the induction of autophagy and protein uptake (Battu et al., 2017). Accordingly, we tested to see if the GCN2 signaling axis was activated by acute arginine withdrawal in previously activated CD8+T lymphocytes. Indeed, many of the downstream targets of the GCN2 pathway, including ATF4, Glutamic-Pyruvic Transaminase 2 (GPT2) and Asparagine Synthetase (ASNS), were up-regulated in arginine-starved CD8+ T cells as detected by RT-PCR (FIG. 1G). Moreover, we also found increased cell-surface expression of the LNAA amino acid transporter CD98 (FIG. 1H) which is known to be transcriptionally regulated by ATF4 (Chen et al., 2014). Taken together, our results suggest that depriving activated CD8+ T cells of arginine leads to activation of the GCN2-ATF4 stress response pathway.

### The GCN2 Agonist Halofuginone Enhances T Cell Effector Function and Oxidative Metabolism

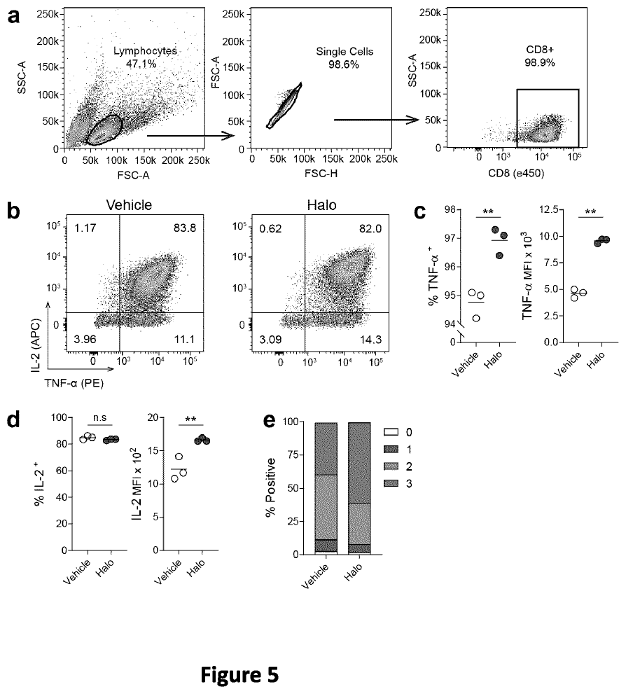

Given that our results indicated that the GCN2 pathway was activated in response to arginine starvation, we tested whether treating activated CD8+ T cells with the GCN2 agonist halofuginone (halo) would similarly enhance effector function and oxidative metabolism. We employed a similar experimental protocol as before in which we activated naïve P14 CD8+ T cells with peptide-pulsed mature dendritic cells for three days followed by an expansion in IL-2 for an additional four days with halo being added for the final 48 hours of culture (FIG. 1I). Using this stimulation protocol, we found that halo-treated CD8+ T cells demonstrated enhanced effector function primarily with increased IFN-γ production (FIG. 1J,K), and to a lesser extent, increased TNF-α and IL-2 production as well (FIG. 5A-D). Halo-treated cells also displayed increased polyfunctionality, as most cells co-expressed all three cytokines suggesting a highly activated phenotype (FIG. 5E). In line with this enhanced activation, we also observed a significant increase in granzyme B expression following treatment with halo (FIG. 1L). Similarly, CD98 expressed was enhanced in response to halo (FIG. 1M). Together, these findings indicate that treatment of effector CD8+ T cells with halofuginone recapitulates the enhanced effector function and CD98 expression observed in arginine starved cells.

Metabolically, similar to the arginine-deprived cells, we found halo treated cells to have increased OXPHOS as evident by an increase in OCR, OCR:ECAR ratio, and ATP (FIG. 1N-P). Given that CD8+ central memory T (Tcm) cells also display an oxidative metabolic phenotype (van der Windt et al., 2012), we investigated if treatment with halo induced any other features of Tcm cells. We found that unlike what has been described for Tcm cells (Pearce et al., 2009; van der Windt et al., 2012) the carnitine palmitoyltransferase-1 (CPT-1) inhibitor etomoxir has minimal effects on OCR in halo-treated cells. (FIG. 1N). These data indicated that the halo-treated cells do not utilize fatty acid oxidation (FAO) to fuel their oxidative metabolic phenotype. Taken together, we demonstrate that the activation of GCN2 within effector CD8+ T cells with halofuginone leads to enhanced effector function and oxidative metabolism.

### Halofuginone Promotes the Transcriptional Regulation of 4-1BB Expression and Mitochondrial Metabolism

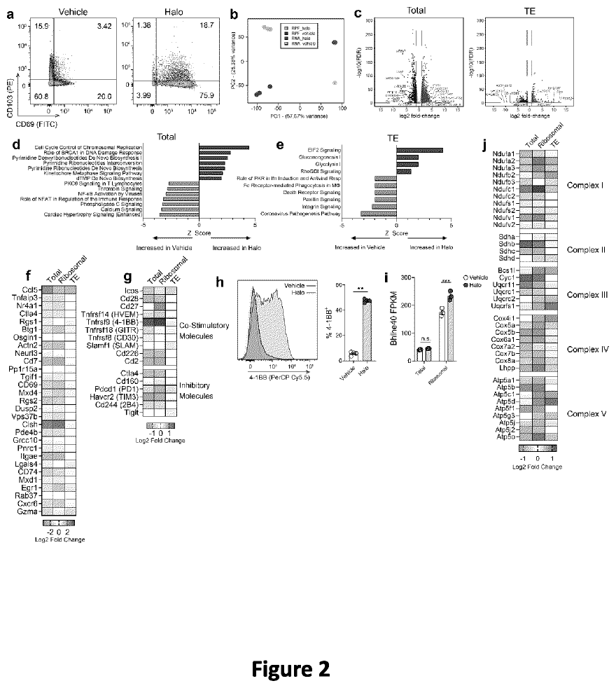

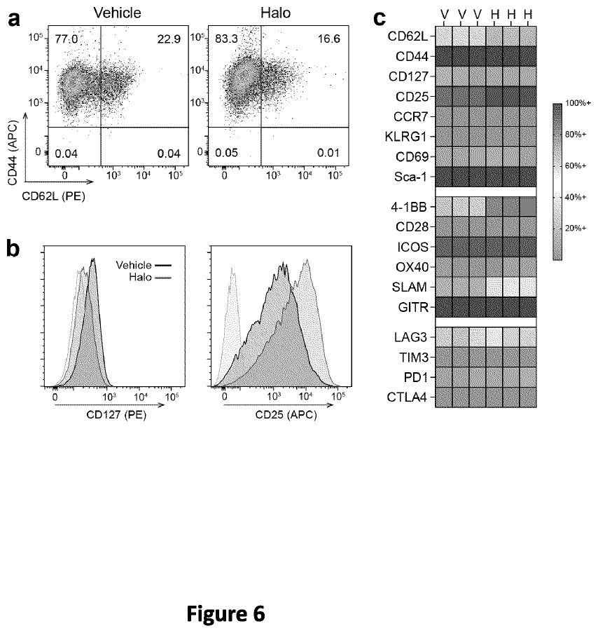

To further support the premise that halo does not induce Tcm cells, we examined the expression of other surface markers associated with different CD8+ T cell lineages. We found that both halo-treated cells and vehicle treated cells were CD62^{Lo}, CD44^{Hi} CD127^{Lo} and CD25^{Hi} which indicates they are not Tcm cells (FIG. 6A,B). Interestingly, unlike vehicle-treated control cells, treating CD8+ T cells with halo induced a population of CD69⁺ CD103⁺ double-positive cells which are suggestive of a tissue-resident memory T cell (Trm) phenotype (FIG. 2A)(Sullivan et al., 2019). To explore the possibility that treatment with halo was inducing Trm cells and to further elucidate the halo-induced phenotype, we performed gene expression profiling using Ribo-Seq analysis (Calviello and Ohler, 2017). We utilized Ribo-Seq as GCN2 is a known regulator of the protein translation response (Battu et al., 2017) and, as such, we hypothesized that treatment with halo might regulate both the cellular transcriptome as well as the translatome. Consistent with this hypothesis, we observed by both principal component analysis (PCA) of all expressed genes and differential analysis (represented by volcano plots) a distinct set of genes enriched in the halo-treated cells versus control cells in both the total RNA pool, and in the translational efficiency (TE), which is a measure of genes regulated at the translational level (FIG. 2B,C). Pathway analysis using ingenuity pathway analysis (IPA) and focusing on the “diseases and functions” results revealed different pathways enriched in the total RNA pool in halo versus vehicle (FIG. 2D) compared to the pathways enriched translationally (FIG. 2E). Not surprisingly, given that halo is known to activate GCN2, the most enriched pathways regulated translationally were related to EIF2 signaling, whilst genes involved in cell cycle and ribonucleotide base synthesis were most affected at the transcriptional level by halo treatment. Collectively, these data indicated that the phenotype of the halo-treated cells could be a result of alteration of both the transcriptional and translational programming of the CD8+ T cell.

To explore if halo treatment was inducing a Trm phenotype, we compared the transcripts that were enriched in the total RNA pool, ribosome-associated pool and those regulated translationally, in the halo-treated cells to a published list of genes associated with the Trm lineage (Kurd et al., 2020). We found that very few of the Trm-associated genes were up-regulated by treatment with halo, either transcriptionally or translationally (FIG. 2F). These data indicated that activation of GCN2 with halo was not inducing the core Trm gene expression program. These data are consistent with a recent report that amino acid deprivation was unable to induce this core Trm gene-expression profile in CD8+ T cells in the lung (Hayward et al., 2020). Of all of the Trm-associated genes, expression of the cellular adhesion molecule and early T cell activation marker CD69 was one of the most upregulated genes by halo treatment (FIG. 2F), This led us to explore the effect of halo on a panel of costimulatory and co-inhibitory molecules associated with T cell activation (FIG. 2G). Expression of most of the markers was not enhanced by treatment with halo at either the transcriptional, translational and protein level (FIG. 2G and FIG. 6C). A notable exception, however, was the co-stimulatory molecule 4-1BB that was up-regulated in halo-treated cells. This enhancement was reflected in the surface expression of 4-1BB, as halo-treated cells demonstrated both an increased percentage of cells expressing 4-1BB as well as a higher level of expression on a per cell basis as assessed by flow cytometry (FIG. 2H). These data, combined with our data on CD69 expression (FIG. 2A) suggested that transcriptional regulation of protein expression may be a significant driver of the observed surface marker phenotype in halo-treated cells.

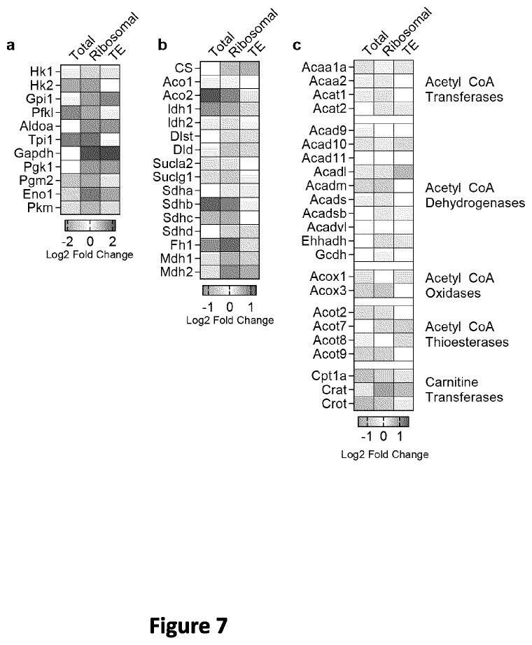

Utilization of an oxidative metabolic phenotype was another central aspect of the observed phenotype of the halo-treated cells (FIG. 1N-P). Accordingly, we investigated if we could find evidence of translationally controlled genes which could drive mitochondrial metabolism in halo-treated cells. Interestingly, halo treatment induced a marked increase in the ribosomal-associated Bhlhe40 transcripts, a transcription factor recently linked to programming mitochondrial metabolism in Trm cells as well as tumor-infiltrating lymphocytes (TILs) (Li et al., 2019) (FIG. 2I). Moreover, halo treatment also primarily increased both transcription and translation of all five complexes of the electron transport chain (ETC) (FIG. 2J). Increased enrichment of genes associated with glycolysis and the TCA cycle, but not fatty acid oxidation, were also found in halo-treated cells (FIG. 7). These data suggest that the GCN2-EIF2 axis has a central role in both transcriptionally and translationally regulating an oxidative metabolic program in activated CD8+ T cells. This program, which involves the expression of Bhlhe40 and proteins involved in the ETC, is similar to the program recently described which was found to be required to support Trm and TIL effector function in the TME (Li et al., 2019).

### Halofuginone Modulates T Cell Function Through Autophagy and the CD98/mTOR Axis

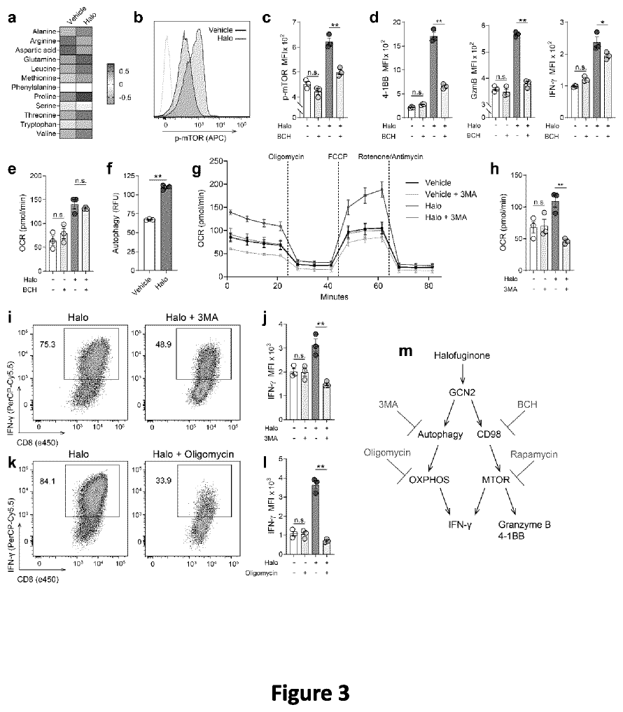

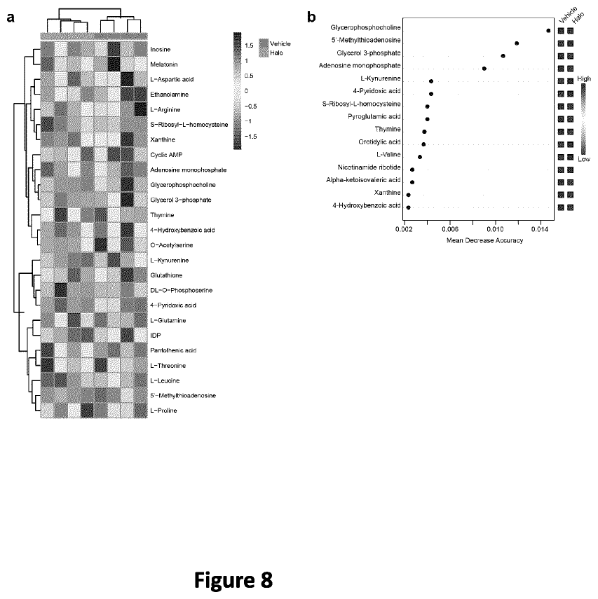

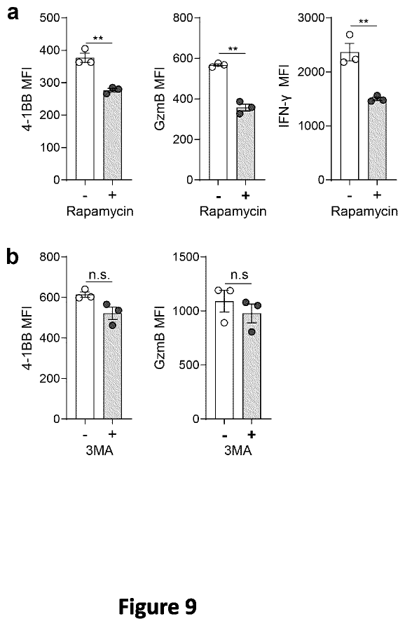

To gain a better insight into the mechanisms mediating the downstream effects of GCN2 activation, we performed targeted mass spectrometry to evaluate the metabolic profile of halo-treated cells (FIG. 8). Many of the metabolic alterations involved levels of amino acids, which were primarily up-regulated in response to halo (FIG. 3A) This correlated with increased expression of CD98 (SLC3A2) that in conjunction with LAT1 (SLC7A5) is a known LNAA transporter (Scalise et al., 2018). In line with previous studies demonstrating a link between CD98-LAT1 and phosphorylation of mTOR in CD8+ T cells (Sinclair et al., 2013), we observed a similar increase in p-mTOR in halo-treated cells (FIG. 3B) which was reversed in the presence of the CD98 inhibitor BCH (FIG. 3C). Furthermore, treatment with BCH also led to an inhibition of expression of 4-1BB, Granzyme B and IFN-γ in halo-treated cells (FIG. 3D). A similar finding was observed when culturing halo-treated cells with rapamycin (FIG. 9A), suggesting that the CD98/mTOR axis regulates the expression of 4-1BB, granzyme B and IFN-γ downstream of GCN2 activation. Interestingly, treatment with BCH did not affect the enhanced OXPHOS observed in halo-treated cells (FIG. 3E) suggesting that the downstream pathway regulating the increased OXPHOS induced by treatment with halo was distinct from the pathway regulating other aspects of the halo phenotype. Given that GCN2 is known to regulate autophagy in other cell types (B'chir et al., 2013), we investigated if treatment with halo induced autophagy in activated CD8+ T cells. Indeed, we detected increased evidence of autophagy in the halo-treated cells as determined by fluorescent staining of autophagosomes (FIG. 3F). These data lead us to hypothesize that autophagy could be the link between the activation of GCN2 and the increased oxidative metabolism observed in the halo-treated cells. To test this, we cultured T cells in the presence of halo and the autophagy inhibitor 3MA. The increased OXPHOS induced by halo was entirely abrogated by blocking autophagy while having no effect on vehicle-treated cells (FIG. 3G,H). This indicated that the increased OXPHOS mediated by GCN2 is largely attributed to autophagy. We also tested whether autophagy can regulate other aspects of the cellular phenotype induced by GCN2 activation. Although we observed no difference in 4-1 BB or granzyme B expression in halo-treated cells cultured in the presence of 3MA (FIG. 9B), we did observe a marked reduction in IFN-γ (FIG. 3I,J). This indicated that autophagy is largely driving both OXPHOS and IFN-γ production. To determine whether it was the autophagy or OXPHOS itself that was responsible for regulating IFN-γ, we cultured halo-treated CD8+ T cells in the presence of the OXPHOS inhibitor oligomycin and again observed a marked reduction in IFN-γ (FIG. 3K,L). These findings indicated that autophagy is regulating IFN-γ through the induction of OXPHOS. Therefore, our findings suggest that the CD98/mTOR and the autophagy/OXPHOS axes work in parallel to mediate the downstream effects of GCN2 activation in effector CD8+ T cells (FIG. 3M).

Halofuginone Synergizes with Immunotherapy to Induce Robust Anti-Tumor Responses

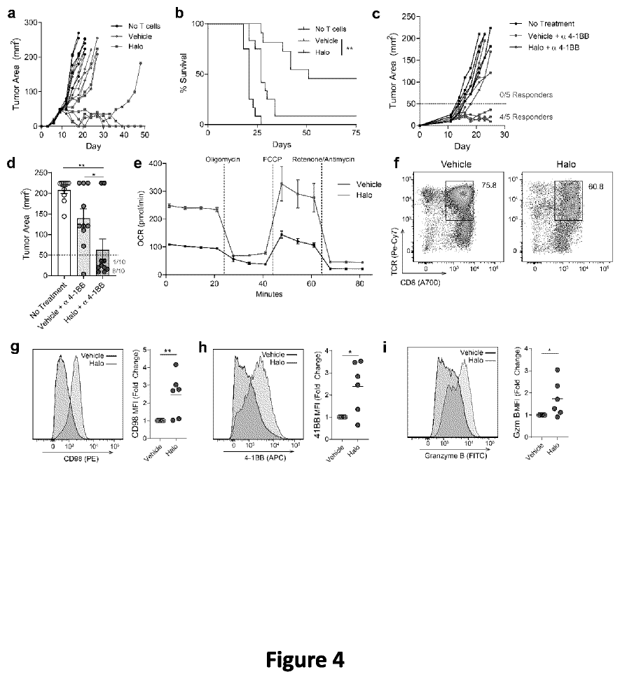

Given that cells with augmented mitochondrial metabolism and IFN-γ production demonstrate enhanced anti-tumor activity (Saibil et al., 2019; Scharping et al., 2016) we evaluated the anti-tumor properties of halo-treated T cells in the context of adoptive immunotherapy. Mice bearing day 10 established EG7-OVA tumors received 1×10⁶ OT-1 CD8+ T cells treated with halofuginone or vehicle control (FIG. 4A). We found that halofuginone increased the anti-tumor properties of T cells and led to curative response in −50% of mice that received halo-treated T cells (FIG. 4A,B). In contrast, over 90% of mice receiving vehicle-treated T cells did not display long-term tumor control. Next, we tested whether halofuginone could synergize with immunotherapy in the B16 melanoma tumor model which is highly resistant to multiple immunotherapeutic modalities. Here, we treated mice bearing established B16gp33 melanoma tumors with sub-therapeutic numbers of halo or vehicle treated CD8+P14 T cells in conjunction with in vivo administration of 4-1BB agonistic antibody. Although mice that received these sub-therapeutic levels of T cells alone did not display any reduction in tumor growth (data not shown), we found that mice that received halo-treated T cells in conjunction with 4-1BB immunotherapy demonstrated robust anti-tumor responses (FIG. 4C). Importantly, 80% of mice demonstrated a response to this combination immunotherapy compared to only 10% of mice that received vehicle-treated T cells in conjunction with 4-1BB immunotherapy (FIG. 4D). Together, these findings indicate that halo treatment of T cells synergizes with agonistic 4-1BB antibody immunotherapy to induce robust anti-tumor responses in immunotherapy-resistant tumors.

### Halofuginone Enhances Metabolism and Effector Function in Human CD8+ T Cells

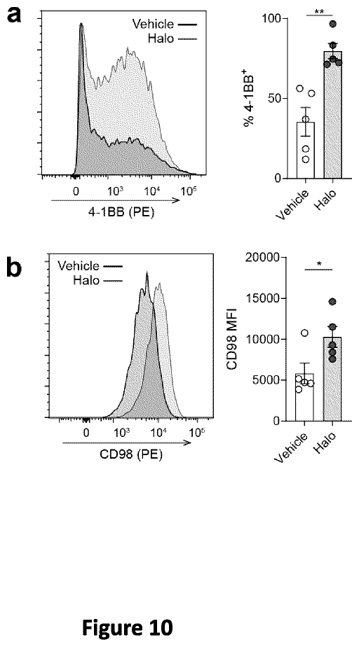

Next, we investigated the effects of GCN2 activation in human CD8+ T cells with halofuginone. Similar to what we found in mice, halofuginone treatment enhanced OXPHOS in addition to increasing the expression of 4-1BB and CD98 on human CD8+ T cells (FIG. 4E and FIG. 10). We then tested whether halofuginone could also be applied to existing clinically relevant protocols involving the transduction of tumor-specific TCRs into human CD8+ T cells isolated from peripheral blood. The addition of halofuginone to the culture conditions did not inhibit transduction efficiency of the DMF5 TCR which recognizes the MART-1 melanoma antigen (FIG. 4F). Furthermore, we found that in the majority of patients, TCR+ T cells activated in the presence of halo had increased expression of 4-1BB, Granzyme B and CD98 mirroring what was observed in mice (FIG. 4G-I). Together, these findings suggest a directly translatable role of halofuginone into existing clinical immunotherapies involving the ex-vivo manipulation and expansion of CD8+ T cells.

The mechanisms by which the tumor micro-environment modulates CD8+ T cell effector function are beginning to be appreciated. Here, we report that CD8+ T cells respond to acute arginine depletion through enhancing oxidative metabolism and T cell effector function which can be recapitulated with the GCN2 agonist halofuginone. Halo treatment lead to alterations in the transcriptome, translatome and metabolome leading to activation of mTOR and autophagy to facilitate the enhanced OXPHOS and effector function. Importantly, halo-treated cells demonstrate robust anti-tumor functions and treatment with halo facilitated the response to 4-1 BB agonistic antibody when combined with adoptive cell transfer in an immunotherapy resistant mouse model. Together, these findings identify the GCN2 pathway and halofuginone as targets to enhance immunotherapeutic protocols.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

B'chir, W., Maurin, A.-C., Carraro, V., Averous, J., Jousse, C., Muranishi, Y., Parry, L., Stepien, G., Fafournoux, P., and Bruhat, A. (2013). The eIF2α/ATF4 pathway is essential for stress-induced autophagy gene expression. Nucleic Acids Res. 41, 7683-7699.
Battu, S., Minhas, G., Mishra, A., and Khan, N. (2017). Amino Acid Sensing via General Control Nonderepressible-2 Kinase and Immunological Programming. Front. Immunol. 8, 1719.
Brand, A., Singer, K., Koehl, G. E., Kolitzus, M., Schoenhammer, G., Thiel, A., Matos, C., Bruss, C., Klobuch, S., Peter, K., et al. (2016). LDHA-Associated Lactic Acid
Production Blunts Tumor Immunosurveillance by T and NK Cells. Cell Metab. 24, 657-671.
Calviello, L., and Ohler, U. (2017). Beyond Read-Counts: Ribo-seq Data Analysis to Understand the Functions of the Transcriptome. Trends Genet. 33, 728-744.
Chang, C. H., Qiu, J., O'Sullivan, D., Buck, M. D., Noguchi, T., Curtis, J. D., Chen, Q., Gindin, M., Gubin, M. M., Van Der Windt, G. J. W., et al. (2015). Metabolic Competition in the Tumor Microenvironment Is a Driver of Cancer Progression. Cell 162, 1229-1241.
Chen, R., Zou, Y., Mao, D., Sun, D., Gao, G., Shi, J., Liu, X., Zhu, C., Yang, M., Ye, W., et al. (2014). The general amino acid control pathway regulates mTOR and autophagy during serum/glutamine starvation. J. Cell Biol. 206, 173-182.
Chong, J., Yamamoto, M., and Xia, J. (2019). MetaboAnalystR 2.0: From Raw Spectra to Biological Insights. Metabolites 9, 57.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Eil, R., Vodnala, S. K., Clever, D., Klebanoff, C. A., Sukumar, M., Pan, J. H., Palmer, D. C., Gros, A., Yamamoto, T. N., Patel, S. J., et al. (2016). Ionic immune suppression within the tumour microenvironment limits T cell effector function. Nature 537, 539-543.
Frey, A. B. (2015). Suppression of T cell responses in the tumor microenvironment. Vaccine 33, 7393-7400.
Hayward, S. L., Scharer, C. D., Cartwright, E. K., Takamura, S., Li, Z.-R. T., Boss, J. M., and Kohlmeier, J. E. (2020). Environmental cues regulate epigenetic reprogramming of airway-resident memory CD8+ T cells. Nat. Immunol. 21, 309-320.
Ingolia, N. T., Brar, G. A., Rouskin, S., McGeachy, A. M., and Weissman, J. S. (2012). The ribosome profiling strategy for monitoring translation in vivo by deep sequencing of ribosome-protected mRNA fragments. Nat. Protoc. 7, 1534-1550.
Klein Geltink, R. I., Edwards-Hicks, J., Apostolova, P., O'Sullivan, D., Sanin, D. E., Patterson, A. E., Puleston, D. J., Ligthart, N. A. M., Buescher, J. M., Grzes, K. M., et al. (2020). Metabolic conditioning of CD8+ effector T cells for adoptive cell therapy. Nat. Metab. 2, 703-716.
Kurd, N. S., He, Z., Louis, T. L., Milner, J. J., Omilusik, K. D., Jin, W., Tsai, M. S., Widjaja, C. E., Kanbar, J. N., Olvera, J. G., et al. (2020). Early precursors and molecular determinants of tissue-resident memory CD8+T lymphocytes revealed by single-cell RNA sequencing. Sci. Immunol. 5, 16-19.
Langmead, B., and Salzberg, S. L. (2012). Fast gapped-read alignment with Bowtie 2. Nat. Methods 9, 357-359.
Lemos, H., Huang, L., Prendergast, G. C., and Mellor, A. L. (2019). Immune control by amino acid catabolism during tumorigenesis and therapy. Nat. Rev. Cancer 19, 162-175.
Leone, R. D., Zhao, L., Englert, J. M., Sun, I.-M., Oh, M.-H., Sun, I.-H., Arwood, M. L., Bettencourt, I. A., Patel, C. H., Wen, J., et al. (2019). Glutamine blockade induces divergent metabolic programs to overcome tumor immune evasion. Science (80-.). 366, 1013-1021.
Li, C., Zhu, B., Son, Y. M., Wang, Z., Jiang, L., Xiang, M., Ye, Z., Beckermann, K. E., Wu, Y., Jenkins, J. W., et al. (2019). The Transcription Factor Bhlhe40 Programs Mitochondrial Regulation of Resident CD8+ T Cell Fitness and Functionality. Immunity 51, 491-507.e7.
Martin, M. (2011). Cutadapt removes adapter sequences from high-throughput sequencing reads. EMBnet. Journal 17, 10.
Najjar, Y. G., Menk, A. V, Sander, C., Rao, U., Karunamurthy, A., Bhatia, R., Zhai, S., Kirkwood, J. M., and Delgoffe, G. M. (2019). Tumor cell oxidative metabolism as a barrier to PD-1 blockade immunotherapy in melanoma. JCI Insight 4.
Ngwa, V. M., Edwards, D. N., Philip, M., and Chen, J. (2019). Microenvironmental Metabolism Regulates Antitumor Immunity. Cancer Res. 79, 4003-4008.
St. Paul, M., Saibil, S. D., Lien, S. C., Han, S., Sayad, A., Mulder, D. T., Garcia-Batres, C. R., Elford, A. R., Israni-Winger, K., Robert-Tissot, C., et al. (2020). IL6 Induces an IL22+CD8+ T-cell Subset with Potent Antitumor Function. Cancer Immunol. Res. 8, 321-333.
Pearce, E. L., Walsh, M. C., Cejas, P. J., Harms, G. M., Shen, H., Wang, L.-S., Jones, R. G., and Choi, Y. (2009). Enhancing CD8 T-cell memory by modulating fatty acid metabolism. Nature 460, 103-107.
Petrova, V., Annicchiarico-Petruzzelli, M., Melino, G., and Amelio, I. (2018). The hypoxic tumour microenvironment. Oncogenesis 7.
Pino, L. K., Searle, B. C., Bollinger, J. G., Nunn, B., MacLean, B., and MacCoss, M. J. (2020). The Skyline ecosystem: Informatics for quantitative mass spectrometry proteomics. Mass Spectrom. Rev. 39, 229-244.
Pircher, H., Bürki, K., Lang, R., Hengartner, H., and Zinkernagel, R. M. (1989). Tolerance induction in double specific T-cell receptor transgenic mice varies with antigen. Nature 342, 559-561.
Renner, K., Singer, K., Koehl, G. E., Geissler, E. K., Peter, K., Siska, P. J., and Kreutz, M. (2017). Metabolic Hallmarks of Tumor and Immune Cells in the Tumor Microenvironment. Front. Immunol. 8, 248.
Robinson, M. D., McCarthy, D. J., and Smyth, G. K. (2010). edgeR: a Bioconductor package for differential expression analysis of digital gene expression data. Bioinformatics 26, 139-140.
Rodriguez, P. C., Quiceno, D. G., and Ochoa, A. C. (2007). 1-arginine availability regulates T-lymphocyte cell-cycle progression. Blood 109, 1568-1573.
Saibil, S. D., St. Paul, M., Laister, R. C., Garcia-Batres, C. R., Israni-Winger, K., Elford, A. R., Grimshaw, N., Robert-Tissot, C., Roy, D. G., Jones, R. G., et al. (2019). Activation of Peroxisome Proliferator-Activated Receptors a and 6 Synergizes with Inflammatory Signals to Enhance Adoptive Cell Therapy. Cancer Res. 79, 445-451.
Scalise, M., Galluccio, M., Console, L., Pochini, L., and Indiveri, C. (2018). The Human SLC7A5 (LAT1): The Intriguing Histidine/Large Neutral Amino Acid Transporter and Its Relevance to Human Health. Front. Chem. 6.
Scharping, N. E., Menk, A. V, Moreci, R. S., Whetstone, R. D., Dadey, R. E., Watkins, S. C., Ferris, R. L., and Delgoffe, G. M. (2016). The Tumor Microenvironment Represses T Cell Mitochondrial Biogenesis to Drive Intratumoral T Cell Metabolic Insufficiency and Dysfunction. Immunity 45, 374-388.
Sinclair, L. V, Rolf, J., Emslie, E., Shi, Y.-B., Taylor, P. M., and Cantrell, D. A. (2013). Control of amino-acid transport by antigen receptors coordinates the metabolic reprogramming essential for T cell differentiation. Nat. Immunol. 14, 500-508.
Sullivan, M. R., Danai, L. V, Lewis, C. A., Chan, S. H., Gui, D. Y., Kunchok, T., Dennstedt, E. A., Vander Heiden, M. G., and Muir, A. (2019). Quantification of microenvironmental metabolites in murine cancers reveals determinants of tumor nutrient availability. Elife 8.
van der Windt, G. J. W., Everts, B., Chang, C.-H., Curtis, J. D., Freitas, T. C., Amiel, E., Pearce, E. J., and Pearce, E. L. (2012). Mitochondrial Respiratory Capacity Is a Critical Regulator of CD8+ T Cell Memory Development. Immunity 36, 68-78.
Werner, A., Koschke, M., Leuchtner, N., Luckner-Minden, C., Habermeier, A., Rupp, J., Heinrich, C., Conradi, R., Closs, E. I., and Munder, M. (2017). Reconstitution of T cell proliferation under arginine limitation: Activated human T cells take up citrulline via L-Type amino acid transporter 1 and use it to regenerate arginine after induction of argininosuccinate synthase expression. Front. Immunol. 8.

1. A method for improving the anti-cancer effect of T-cells, the method comprising:
providing a population of T-cells; and
culturing the T-cells in an environment that activates the GCN2 pathway.

2. The method of claim 1, wherein the environment includes a GCN2 pathway agonist.

3. The method of claim 2, wherein the GCN2 pathway agonist is a tRNA synthetase inhibitor.

4. The method of claim 3, wherein the GCN2 pathway agonist is halofuginone.

5. The method of claim 3, wherein the GCN2 pathway agonist is selected from febrifupene, MAZ1310, MAZ1442, halofuginol, [³H]-halofuginol([³H]-5), epi-febrifuginol, and 5′-O—[N-(L-prolyl)-sulfamoyl]adenosine.

6. The method of claim 1, wherein the GCN2 pathway agonist is added to the culture immediately following isolation of the T-cell population.

7. The method of claim 1, wherein the GCN2 pathway agonist is added to the culture within 2 weeks following isolation of the T-cell population.

8. The method of claim 1, wherein the environment is amino acid deficient or depleted.

9. The method of claim 1, wherein the T-cells are CD8+.

10. The method of claim 1, wherein the T-cells are a Tumour Infiltrating Lymphocytes.

11. The method of claim 1, wherein the T cells express chimeric antigen receptors (CARs).

12. A population of anti-cancer T-cells produced by the methods of claim 1.

13. (canceled)

14. (canceled)

15. A method of treating a patient with cancer, the method comprising administering to the patient the population of anti-cancer T-cells of claim 12.

16. A method of treating a patient with cancer, the method comprising administering to the patient a GCN2 pathway agonist.

17. The method of claim 16, wherein the GCN2 pathway agonist is a tRNA synthetase inhibitor.

18. The method of claim 17, wherein the GCN2 pathway agonist is halofuginone.

19. The method of claim 18, wherein the GCN2 pathway agonist is selected from febrifugene, MAZ1310, MAZ1442, halofuginol, [³H]-halofuginol([³H]-5), epi-febrifuginol, and 5′-O—[N-(L-prolyl)-sulfamoyl]adenosine.
